(19) United States
(12) Patent Application Publication
(10) Pub. No.: US 2019/0008450 A1
(43) Pub. Date: Jan. 10, 2019

(54) APPARATUS AND METHOD FOR EARLY DETECTION, MONITORING AND TREATING SLEEP DISORDERS

(71) Applicant: SLEEPOW LTD., Hod Hasharon (IL)

(72) Inventors: Yacov GURIEVSKY, Hod Hasharon (IL); Mordehai RATMANSKY, Tel Aviv (IL)

(21) Appl. No.: 16/067,658

(22) PCT Filed: Dec. 29, 2016
(86) PCT No.: PCT/IL2016/051402

§ 371 Date: Jul. 2, 2018

Related U.S. Application Data
(60) Provisional application No. 62/272,985, filed on Dec. 30, 2015.

(51) Int. Cl. A61B 5/00; A61B 5/0205; A61M 21/02; A61B 5/117; A61B 90/50

(52) U.S. Cl. CPC A61B 5/4812; A61B 5/6833; A61B 5/02055; A61B 5/6823; A61B 5/721; A61B 5/7282; A61B 5/02416; A61B 5/4836; A61B 5/684; A61B 5/117; A61B 90/50; A61B 5/0002; A61M 21/02

(57) ABSTRACT

Devices for monitoring one or more parameters of a subject, and related methods and systems, are disclosed. The device comprises a support element configured and arranged to detachably attach over a skin region of a chest area of a subject, an optical sensor assembly mounted to said support element and configured and arranged to conduct optical measurements in said chest area and generate optical measurement data indicative of said one or more parameters of said subject, a volumetric sensor assembly mounted to said support element and configured and operable to measure chest expansions and retractions of said subject and generate volumetric measurement data indicative of the expansions and retractions of the chest of said subject, and a control unit mounted to said support element and configured and operable to process said optical and volumetric measurement data for removing interferences introduced into said optical measurements due to said chest expansions and retractions.

### TECHNOLOGICAL FIELD

The present invention is generally in the field of sleep monitoring, and particularly relates to monitoring sleep, sleep patterns and to diagnosing and treating sleep disorders.

### BACKGROUND

Recent studies and research report a direct link between obstructive sleep apnea (OSA), Insomnia and neurological, mental and physical disorders, disease and illness. According to the CDC since 2007—Sleep deprivation is an official epidemic in the USA. According to estimations OSA effects 10% of the adult population.

Some of the sleep monitoring and treatment solutions proposed in patent literature are briefly described hereinbelow.

US Patent Publication No. 2002/0165462 describes a technique for collecting and analyzing physiological signals to detect sleep apnea, utilizing a small light-weight physiological monitoring system affixed to a patient's forehead to detect and record the pulse, oximetry, snoring sounds, and head position of a patient to detect a respiratory event, such as sleep apnea. The physiological monitoring system may contain several sensors including a pulse oximeter to detect oximetry and pulse rate, a microphone to detect snoring sounds, and a position sensor to detect head position. The physiological monitoring system also can contain a memory to store or record the signals monitored by the mentioned sensors and a power source. The physiological monitoring system may be held in place by a single elastic strap, thereby enabling a patient to use the system without the assistance of trained technicians.

US Patent No. 2008/0308112 discloses a system and method for reducing snoring and sleep apnea of a sleeping person, the system comprising at least one sensor for detecting occurrence and/or likeliness of occurrence of the snoring and/or sleep apnea and for producing a sensor signal indicative of the occurrence and/or likeliness of the occurrence of the snoring and sleep apnea and a processor unit for determining from the sensor signal whether the occurrence of the snoring and/or sleep apnea of the sleeping person is likely, wherein the system is provided with a stimulator controllable by the processor unit, wherein the stimulator is arranged to trigger the sleeping person to change position using a triggering signal.

US Patent Publication No. 2011/132378 describes systems and methods for controlling the position of a user of a wearable positional therapy device configured to monitor and store physiological signals that can be used to assess sleep quality and sleeping position of a user. The device can be configured to be worn around the head, the neck, or body of the user. The device can be configured to provide feedback to a user if the user is sleeping or is positioned in a target position to induce the user to change positions. The feedback can be provided by one or more haptic motors that can be configured to provide various levels of feedback and the level of feedback can be customized based on the user's reaction to the feedback.

US Patent Publication No. 2014/0228711 describes a device for sleep apnea avoidance and data collection using a sensor configured to sense a pressure and generate a first signal when the pressure exceeds a threshold. A signal generator module may be configured to generate a first stimulating signal in response to the first signal. The sensed pressure may include a pressure exerted on the sensor when a user of the device lies down on the device. The first stimulating signal may be configured to cause the user to change sleeping position, for example, from a first sleeping position that causes snoring to a second sleeping position that stops snoring.

US Patent Publication No. 2016/0007914 describes systems, apparatus, and methods of monitoring and reducing snore are discussed herein. Some embodiments may provide for a system including a snore detection module, a movement detection module, a control module, and an actuation module. The snore detection module may be configured to detect snore, such as by detecting vibrations caused by snoring. When snoring is detected, the control module may be configured to instruct the actuation module to apply stimulation to the user that is calibrated to cause the user to shift sleeping position without disturbing sleep. The movement detection module may be configured to monitor user movement. If the user fails to move in response to the actuation, the actuation module may increase the intensity of the actuation. If the user responds to the actuation, the process may be repeated after a predetermined delay to provide continuous snore monitoring and correction throughout user sleep.

### GENERAL DESCRIPTION

Disclosed are technologies for the in-house monitoring of sleep patterns of a user, and for the identification and treatment of sleep disorders, such as, but not limited to, apnea and insomnia. Most of the solutions for the diagnosing and treating of sleep disorders suggested heretofore utilize a wearable device designed for placement on head or neck areas of a user in proximity to the respiratory pathways for detecting, recording and/or processing, snoring sounds of the examined user, and detection of sleep disorders therefrom. The inventors hereof realized that accurate monitoring of user's sleep patterns, and the detection of sleep disorders, can be efficiently and effectively carried out by measuring various physiological and/or biological parameters from a chest area of the user, without causing any discomfort and affecting user's sleep quality.

The present application discloses sleep monitoring devices designed to be removably attached to a chest area of a user, accurately measure various body physiological and/or biological parameters and conditions therefrom, and identify and treat sleep disorders in real-time. For this purpose the inventors hereof developed techniques for accurately measuring various physiological/biological parameters and/or conditions from a chest area of a user, without affecting the user's sleep, by effectively removing interferences from the measured data.

System and methods are described for diagnosing and treating sleep disorders, particularly useful for obstructive sleep apnea (OSA). In some embodiments a feedback loop is used to periodically (or intermittently) measure various physiological parameters of a sleeping user and generate respective measurement data indicative thereof. The generated data is processed and analyzed and sleep patterns of the user are evaluated based on the generated measurement data. Whenever the measurement data is indicative of a sleep disorder, a corresponding treatment session is determined based thereon, and applied to the user until it is apparent from the newly generated measurement data that the diagnosed sleep disorder has lapsed and a healthy sleep pattern been reached.

The system may comprise various sensing elements and circuitries structured and arranged together with auditory/olfactory/electromechanical means for easy attachment to a chest area of the user, and configured and operable to measure, inter alia, oxygen saturation (expressed as a percentage of the maximal binding capacity of oxygen to hemoglobin), breath rhythm, heart pulse, chest expansion, breathing voices (e.g., snoring), user's body position, and/or body temperature, and apply one or more treatment sessions. The treatment sessions can include stimulus, such as producing audible signals, vibrations, skin contact and/or tingling, and/or odors. The treatment sessions applied to the user are designed to cause the user to change sleep position/posture while maintaining a continuous sleep state of the user, or temporarily causing a light sleep state, without awakening the user. If the user awakes during the treatment session, then a sleep aiding session is applied until sleeping patterns are restored.

In possible embodiments the system further utilizes various auxiliary units configured and operable to facilitate change of the user's body position and/or alter user's sleep state (e.g., specially designed pillow and/or mattress, and/or covers for the same, robotic arm, and suchlike).

In some embodiments a pillow cover and/or head support unit embedded with sensors are used for monitoring the user's sleep pattern and for identifying and treating sleep disorders. Optionally, and in some embodiment preferably, the pillow cover and/or head support unit sensors comprises sensor(s) configured to identify the presence and orientation of user's head with respect to the head support, and generate output data/signals indicative thereof. The sensor(s) can be configured for sensing biological and/or physiological parameters, such as, but not limited to, pulse rate, breathing rate, brainwaves, and suchlike. Acoustic sensor(s) may be sued for identifying sounds from the user's body. The measurement data/signals generated by one or more of the sensor(s) of the pillow cover and/or the head support unit are used in some embodiments for detecting and treating sleep disorders such as OSA and/or insomnia.

The monitoring device can be used for purpose of diagnostic, monitoring, therapeutic, behavioral, sleep behavior, education, and/or sleep and wakefulness states education. In some embodiments the monitoring device is configured and operable to communicate data with the pillow cover and/or head support unit, and process the measurement data/signals received therefrom to determine sleep patterns of the user and/or sleep disorders of the examined user. Accordingly, one or more processors and memory units (volatile and non-volatile) can be used in the monitoring device to communicate, record and/or process the measurement data collected by the various sensors of the system. Optionally, one or more processors and memory units (volatile and non-volatile) are used by the pillow cover and/or head support unit to process and/or record the collected measurement data.

In some embodiments the pillow cover and/or head support unit and/or mattress and/or mattress cover also comprise one or more actuators configured and operable to apply one or more stimulations for causing the user to change position, preferably without awaking the user. The monitoring device can be thus configured to process the measurement data and actuate one or more actuators whenever sleep disorders are determined. Intensity and/or pattern (number of repetitions and/or repetition frequencies) of the stimulation(s) to be applied can be also determined by the monitoring device based on the sleep disorders determined from the measurement data.

Audio output devices can be provided in the pillow cover and/or head support configured to generate an audio binaural patterns. Optionally, and in some embodiments preferably, the audio binaural patterns are structured such that the audio signals perceived by the left and right ears of the user are substantially of the same magnitudes but having different spectral components. For example, and without being limiting, in some embodiments the audio binaural patterns have a frequency difference of about 0.5 Hz between the audio signals generated for the left and right ears of the user (i.e., there is a frequency difference between the audio signals outputted by the left and right speakers), to thereby affect a desired state of mind. Moreover, the audio binaural patterns can be selectively generated, upon identifying certain conditions and/or events from the sensors' data. For example, the audio binaural patterns can be induced/applied whenever certain sleep patterns and/or disorders are detected, and removed/stopped whenever healthy sleep patterns are determined from the measured data/signals. Any musical piece/tune can be used for generating the audio binaural patterns. In some embodiments a playlist of musical pieces selected by the user is used.

Optionally, and in some embodiments preferably, the a periodical pulsating audio signal (also referred to herein as beats) is generated and played to the user. The frequency of generated beats can be changed during a treatment session, by initially generating beat signals at frequencies slightly greater or smaller than the heart rate of the user, and thereafter progressively reducing the frequency of the generated beat signals towards a heart rate frequency of the user. In some embodiments the beat signals are initially generated at frequencies in the range of 70-80 Hz and progressively/monotonically reduced to about 50-60 Hz. The generated beat signals can be also adapted to provide a binaural patterns played to the left and right ears of the user with a frequency difference of about 0.5 Hz. The audio beat signals can be combined into any other audio signal/musical piece played to the user by the system during a treatment session.

Accordingly, the monitoring device, and/or the pillow cover and/or head support unit, can be configured to introduce into the audio patterns binaural beats at predefined rates for inducing a desired sleep state and state of mind. The sensor elements installed in the head support unit are used to measure various biological properties of the user's body (e.g., heat rate, respiratory rate, body position, and/or brainwaves and other physiological parameters), and adjust the audible signals/binaural beats accordingly. This way, biofeedback processed can be carried out by adjusting the frequency of the audible signals/binaural beats according to the parameters measured from the user's body, for example, according to the measured heart rate, and/or respiratory rate. This way the system can be engineered to adjust the frequency of the generated beat signals to be constantly smaller by few Hertz from the heart pulse rate of the user, for example about 1 to 5 Hz smaller than the measured heart pulse of the user, and to gradually converge towards the frequency of the user's heart pulse rate in a sleeping state.

Accordingly, methods are provided for adding binaural beats by conversion of audio signals (e.g., user selected tunes) based on heart and/or respiratory rate measured from the user's body, thereby creating an updated audio stream with an extra sound channel. In this way, original audio signals/sound tracks can be modified to apply binaural beats synchronized with heart rate/beats and/or respiratory rate, in real time on one soundtrack.

In some embodiments, the monitoring device comprises a bone conduction unit configured and operable to mechanically deliver signals to the body of the user. The bone conduction unit can be implemented using a flat sheet attached over the a skin area on top of the bone and a vibration generating device mechanically coupled thereto and configured to deliver vibrations to the bone via the flat sheet in a defined frequency and/or resonance. Optionally, a diffuser is used for inducing medicaments/drugs and/or scents into the user's environment (e.g., by evaporation and/or spraying). The diffuser can be provided in the monitoring device, and/or the pillow cover, and/or the head support unit, and/or other auxiliary elements of the system (e.g., robotic arm), and/or as an independent separate unit placed in a vicinity of the user.

In order to maintain stereo effect and effectively endorse sleep state alternation, three or more speakers are used in the system. The speakers can be embedded or inserted in the pillow cover and/or the head support unit and/or the bed. Optionally, and in some embodiments preferably, the speakers are hidden seamlessly in one or more of parts of the system e.g., located under foam and fabric. In some embodiments the system is configured to selectively switch one or more of the speakers between operating and non-operating states based of the measurement data.

For example, and without being limiting, in some embodiments one or more accelerometers and/or displacement sensors are provided in the monitoring device attached to the user's body for determining sleep position of the user. In the back sleeping position stereo effect is optimal, but when the user turns to a side sleep position stereo effect is no longer maintained effectively. Therefore, the monitoring device is configured in some embodiments to deactivate a left, central, and/or right speaker(s), out of the three or more speakers, if it is determined that the user is positioned on the back, or turns to sleep over one side, or turns into the abdominal position, in order to maintain stereo effect on user. In this way, three or more speakers can be used to maintain stereo left/right audio perception by the user, regardless of the sleeping position of the user.

In some embodiments the monitoring device is used to apply psychological therapy sessions utilizing a personal artificial intelligence (AI) based on coaching assistant module. The AI module can be associated with the a specific examined user for providing psychical guidance support e.g., mimicking a Therapist, Teacher, Guru, Master, or mentor, or suchlike. The AI module can utilize learning processes/algorithms for collecting and classifying the collected measured data according to sleep states and/or sleep disorders of the user's, which can be then used to diagnose newly collected measured data based on similarity criterions. The AI module can be also configured to guide the user by vocal guidance and/or visual commands, and various stimulations that can be applied by the system.

Optionally, and in some embodiments preferably, a computerized device, such as a desktop/laptop computer, tablet, and/or smartphone, are used for filling a questioner (e.g., utilizing icons, questions, videos, images) for receiving personal information from the user. The information obtained via the questioner can be transferred to the monitoring device via any suitable wired (e.g., USB connection) and/or wireless (e.g., WiFi, ZigBee, Bluetooth, NFC, IR and/or sonic/ultrasonic signaling, or suchlike) data communication link.

The filled questioner can be also sent to a remote data base and/or monitoring center, to thereby enable connecting between psychological conditions and facilitating achieving user's sleep goals. In some embodiments CBTi (cognitive behavior therapy insomnia) techniques are utilized (e.g., in case insomnia is diagnosed by the monitoring device), positive psychology, Mantra's, Specialty Treatment programs, guided imagery, hypnotic scripts, Affirmations and/or any type of audiovisual treatment session, is used to provide guidance, instructions/commands, and/or support to the user. One or more communication modules can be used in the monitoring device attached to body of the user to enable it to communicate data with and over a data network, such as, but not limited to, the Internet, cellular networks, satellite networks, Bluetooth networks, LANs, WLANs, and any combination thereof. This way, the monitoring device can be configured to exchange data with remote manned, or un-manned, monitoring center(s) having expert data and/or professional personals trained to provide one or more treatment session for providing the user the needed guidance and/or support.

These treatment sessions can be carried out while the user is awake or during sleep states, or in between sleep and wake states. The guidance, instructions/commands, and/or support provided to the user can be directed to specific matters, such as mental disorders or conditions, based on clinical protocols maintained in the remote AI module/database and/or monitoring center.

Whenever sleep disorders, such as OSA, are identified in the determined sleeping pattern(s) the monitoring device generated control signals for applying one or more sensory, audio and/or visual stimulation by one or more actuators, for causing the user to change the sleep position, and/or slightly awake the user. The one or more actuators are configured in some embodiments to apply one or more audio signals/sounds, one or more scents, light effects (e.g., by LEDs), vibrations (e.g., by vibrating motor and/or robotic arm when identifying OSA), change of mattress position and/or mattress angel. Additionally or alternatively, the monitoring device can be configured to activate a smart home system and/or a wireless telemedicine system, whenever sleep disorders are identified, for causing the user to change sleep position and/or enter healthy sleep state.

Optionally and in some embodiments preferably, the systems is configured to cause the user to change position/posture from the back sleeping position (about 90° turn to the side) and thereby allow user's air ways to naturally open. In case sleep disorders, such as OSA, are identified, and applied audio signals were not effective in re-positioning the user's sleep posture, the system can activate the AI module that decides whether to increase volume level and or change the sounds types/tunes played and combine binaural beats and/or vibration feedback, scents, light feedback, bed angle position, and suchlike, in order to stop the OSA incident without waking up the user.

The AI module can further employ machine learning processes/algorithms to determine the effectiveness of each applied stimuli (e.g., sound, tactile vibration, light, scent, and suchlike) in changing the sleeping position and/or patterns of the user, without fully awaken the user. In some embodiments the machine learning processes/algorithms is configured to constantly apply stimulations while changing parameters of the applied stimulation (e.g., frequency, volume, melody, pitch, vibration force, lumens level etc.), and monitor and determine the user's response to the applied stimulation in real time, and identify working stimuli combinations and the parameters of each stimuli that managed to affect the required change in the user's sleeping position/pattern. This way, the more the monitoring device is used more input data is generated via the system's sensors for the machine learning processes/algorithms.

In this way, whenever a certain sleep disorder is identified, such as Insomnia or OSA, a previously selected stimuli combination and its effective parameters (e.g., comprising actuation of one or more different actuators) is applied based on the results obtained by the machine learning process/algorithm. In case a stimuli was not effective in changing the user's sleep disorder and/or sleeping pattern (e.g., OSA or insomnia) incident the system will activate in real time an alternate stimuli program set comprising a different stimuli combinations and/or using different activation parameters.

In case the user becomes less responsive, or unresponsive, (i.e., tolerant) to the stimulations that were found to be effective in previous treatment sessions in causing change of sleep position and/or patterns, the monitoring device will automatically detect that the user became tolerant to a specific stimulation, or to a combination of stimulations as time elapses without change in the sleep position/pattern, and will correspondingly choose an alternative stimuli program (referred to herein as anti-conditioning).

Following several consecutive uses the monitoring device will identify the parameters of the user's sleep disorders, such as OSA patterns, and will devise corresponding treatment sessions/stimulation combinations for further prevention and treatment of such disorders.

In some embodiments the monitoring device is a personal home sleep test device configured for in-house use. Optionally, the device comprises an air flow sensor configured to measure air flow rates in the user's respiratory system and generate data indicative thereof, a Spo2 (% oxygen in blood) measurement unit configured and operable to measure oxygen saturation in the user's blood and generate data indicative thereof, heart pulse measuring unit configured and operable to measure the user's heart rate and generate data indicative thereof, a movement/displacement sensing unit configured and operable to measure movement of the user's body and generate data indicative thereof, and a microphone configured and operable for measuring auditory signals from the body of the user and generate data indicative thereof (this configuration is also referred to herein as p-HST implementation).

This device can be implemented in a form of a bracelet (also referred to as a p-HST bracelet), for simplicity of design and functionality. This implementation can be designed for mass manufacture of disposable monitoring devices, such as a patch that can be disposed of after several uses/nights. Since the data collected by the device about the user's sleeping patterns, sleep disorders, and the working stimulations of the user, is transferred to a remote database and/or monitoring center, whenever a new monitoring device is used by user, the data collected and recorded at the remote database/center can be downloaded through the data network into the new device, before commencing use thereof. Alternatively, or additionally, the data collected by a formerly used monitoring device can be transferred directly, over wired (e.g., USB, UART) and/or wireless (e.g., WiFi, ZigBee, BlueTooth, NFC, IR and/or sonic/ultrasonic signaling) data communication link, to the new device before commencing use thereof.

In a similar fashion, the monitoring device can be implemented as a wearable article i.e., a dressing item such as a shirt or undershirt, configured with one or more (or all) of the sensing device/units described hereinabove and hereinbelow, a control unit for processing measured data generated by the various sensors and determining sleep patterns and/or disorders, and optionally one or more actuators for applying stimulations whenever the control unit determines that the determined sleep disorders require intervention.

The monitoring device disclosed herein can be alternatively implemented in a form of a pillow, a pillow cover, a mattress, a mattress cover/protector, HST Bracelet, position change detection device, a CBTi device, or a Binaural device.

Optionally, the monitoring system/device is configured to determine sleep architecture from the measurement data obtained from the sensors, by processing with specialized algorithms to provide an output that describes the sleep patterns (REM, non-REM cycles and sleep efficiency). Additionally or alternatively, the system/device is configured to determine user's sleep pathologies, such as sleep apnea and suspected insomnia, and/or environment influence such as noises. The system/device can be configured and operable to measure electrocardiogram of the subject using EMG sensors/electrodes optionally provided in the monitoring device, in a shirt/undershirt, a mattress cover, or suchlike.

Optionally, the monitoring device/system comprises one or more biosensors (e.g., embedded in the monitoring device, and/or the mattress, and/or the pillow cover, and/or shirt/undershirt) configured and operable for measuring biochemical components in the skin, or through the skin, or in the blood, of the user e.g., hormones, and/or neurotransmitters and/or toxins, and/or drugs, and/or pathogens, and/or any precursor of molecules of these biochemical components and/or any degradable molecule that is the result of these biochemical components.

One inventive aspect of the subject matter disclosed herein refers to a device for monitoring one or more parameters of a subject. The device comprising a support element configured and arranged to detachably attach over a skin region of a chest area of a subject, an optical sensor assembly mounted to the support element and configured and arranged to conduct optical measurements in the chest area and generate optical measurement data indicative of the one or more parameters of the subject, a volumetric sensor assembly mounted to the support element and configured and operable to measure chest expansions and retractions of the subject and generate volumetric measurement data indicative of the expansions and retractions of the chest of the subject, and a control unit mounted to the support element and configured and operable to process the optical and volumetric measurement data for removing interferences introduced into the optical measurements due to the chest expansions and retractions.

The device can comprise a displacement sensor assembly configured and operable to measure displacements of the device and generate displacement measurement data indicative thereof. Optionally, and in some embodiment preferably, the control unit is configured and operable to determine breath rhythm of the monitored subject based on at least one of the displacement measurement data and the volumetric data.

Optionally, the one or more parameters comprises oxygen saturation. The control unit cab be configured and operable to determine heart rate of the subject based on the optical measurement data. The device can be also configured and operable to measure respiration or breathing rate.

In some embodiment a sound measurement assembly mounted to the support element is used to measure respiratory sounds of the subject and generate data indicative thereof. The control unit can be configured and operable to determine sleep, sleep architecture, sleep pathologies, environment influence. Optionally, the displacement sensor assembly comprises an accelerometer.

A stimulation arrangement mounted to the support sheet is used in some embodiments to apply at least one stimuli to the subject based on determined sleep state and/or pattern. The stimulation arrangement can comprises at least one of the following: electrodes mounted to the support element and configured and arranged to apply electrical stimulation to the skin region of the subject electromechanical assembly mounted to the support element and configured and arranged to apply vibrations to the skin region of the subject signals generator mounted to the support element and configured and arranged to produce auditory signals; an evaporator and/or diffuser mounted to the support element and configured and arranged to discharge one or more odorants and/or drugs and/or visual stimulation. Optionally, and in some embodiments preferably, the stimulation arrangement comprises a mechanical actuator assembly mounted to the support element and configured and arranged to apply tactile pressure or vibratory stimulations.

In some embodiment a position sensor assembly mounted to the support sheet is used to generate position measurement data indicative of a body position of the subject. Optionally, the control unit is configured and operable to identify sleep disorders or sleep architecture and responsively use the stimulation arrangement to apply one or more stimuli to the subject. The control unit can be configured and operable to apply the one or more stimuli until the position measurement data is indicative of a change in a body position of the subject.

The audio signals generator can comprise at least two speakers. The control unit can be configured and operable to cause the audio signals generator to produce directional auditory stimuli to cause the subject to change body position.

The control unit can be configured and operable to adjust at least one of frequency and magnitude of at least one of the applied stimuli according to the measured data and guarantee that a continuous sleep state of the subject is maintained, or to cause a light sleep state thereof without awakening the subject. Optionally, the control unit is configured and operable to synchronize between at least one of the measured parameters and at least one of the applied stimuli.

In some embodiment the control unit is configured and operable to apply the one or more stimuli until identifying that the sleep disorder has lapsed and a healthy sleep pattern been established. The control unit can be configured and operable to use the stimulation arrangement to apply sleep aiding stimulations whenever identifying that the subject awakened until sleeping patterns are restored.

Optionally, the device is coupled to an external actuator configured and operable to mechanically move one or more regions and/or organs of the subject to thereby affect a change in the body position of the subject, and wherein the control unit is configured to actuate the external actuator whenever sleep disorders are identified. The external actuator can comprise at least one robotic arm.

The device comprises in some embodiments a communication unit configured and operable to exchange data and/or instructions with the device via RF and/or sonic/ultrasonic and/or optical data communication. Optionally, the control unit is configured and operable to use the communication unit to communicate data over a data network with at least one remote computer device or server. The control unit can be configured and operable to use the communication unit to communicate data with at least one user device.

In a variant, the control unit is configured and operable to carry out calibration of the device based on data inputs received from the subject via the communication unit. Optionally, the data inputs comprise at least one of the following: subject's weight, subject's height, subject's BMI, subject's skin color, subject's age, subject's geographic location, subject's gender, subject's general ethnic classification, subject's body fat mass. The calibration can comprise adjusting operational parameters of the optical sensor assembly based on the received data.

In some embodiment the comprises at least one imaging unit configured and operable to generate imagery data of the subject. Optionally, and in some embodiments preferably, the control unit is configured and operable to receive and process imagery data received from at least one of the imaging unit and/or the at least one user device and determine the location of the device based thereon. The control unit can be configured and operable to determine location of the device relative to upper sternum of the subject, or to anterior chest on the left side below the pectoralis muscle. This way, the control unit can be configured and operable to generate directions instructing precisely relocating the device over a desired are of the chest of the subject.

In some embodiments the device comprises a temperature measurement unit configured and operable to measure body temperature of the subject and generate temperature data indicative thereof.

The control unit can be configured and operable to determine based on the measured data at least one of: Spo2 of the subject, electromyogram and/or electroencephalogram of the subject.

In some embodiments the device comprises one or more biosensors configured and operable for measuring biochemical components in the skin, or through the skin, or in the blood, of the subject. Optionally, the biochemical components comprise at least one of the following: hormones; and/or neurotransmitters; and/or toxins; and/or drugs; and/or pathogens; and/or any precursor of molecules of these biochemical components and/or any degradable molecule that is the result of these biochemical components.

At least one biometric sensor arrangement can be used in the device to measure at least on biometric property of the subject and generate data indicative thereof. This way, the control unit can be configured and operable to identify the subject based on the data generated by the at least one biometric sensor arrangement.

Optionally, the control unit is configured and operable to determine at least one of apnea hypopnea index (AHI) and respiratory disturbance index (RDI) of the subject based on the measured data.

Another inventive aspect of the subject matter disclosed herein relates to a method of monitoring sleep state of a subject, comprising: measuring optical data indicative of passage of light through tissue at a chest area of the subject, measuring tension data indicative expansions and/or retractions of the subject's chest, and processing the optical and tension data to determine a sleep pattern and/or state of the subject. The method can comprise processing the tension to identify interferences induced in the measured optical data, and manipulating the optical data to minimize effects of the interferences thereon. Optionally, the measured optical data is process to determine at least one of blood Oxygen saturation, breathing rate, and heart rate. Additionally or alternatively, the measured tension data is processed to determine at least one of breathing rate of the subject, chest expansion events, and chest retraction events. The method can comprise measuring at least one sound data indicative of sounds generated by the subjects, imagery data of the body of the subject, temperature data indicative of body temperature of the user.

Optionally, and in some embodiments preferably, the method comprises processing the measured data to determine one or more sleep patterns of the subject, and applying one or more stimulations whenever identifying unhealthy sleep patterns in the determine one or more sleep patterns. Optionally, the one or more stimulations comprises at least one of binaural audio signal and beats.

A yet another inventive aspect of the subject matter disclosed herein relates to a system for monitoring sleep of a subject. The system comprising a monitoring device configured and arranged to attach to a chest area of the subject, the monitoring device being configured and operable to measure optical data and tension data from the chest area of the subject and determine at least one sleep pattern of the subject based thereon, and at least one stimulation applicator configured and operable to apply one or more stimulations to the subject whenever unhealthy sleep patterns are identified. In some embodiments the at least one stimulation applicator comprises at least one of three or more speakers, one or more robotic arms, one or more electrodes, one or more vibrators, one or more diffusers, and an actuable mattress. The actuable mattress can comprise a movable head support section and an actuator configured and operable to apply rotary movement to the head support section.

### BRIEF DESCRIPTION OF THE DRAWINGS

In order to understand the invention and to see how it may be carried out in practice, embodiments will now be described, by way of non-limiting example only, with reference to the accompanying drawings. Features shown in the drawings are meant to be illustrative of only some embodiments of the invention, unless otherwise implicitly indicated. In the drawings like reference numerals are used to indicate corresponding parts, and in which:

FIG. 1 is a block diagram schematically illustrating a monitoring device according to some possible embodiments;

FIG. 2 is a block diagram schematically illustrating another possible embodiment of the monitoring device;

FIG. 3A to FIG. 3C schematically illustrate possible embodiments of the monitoring device implemented in a form of an adhesive patch, wherein FIG. 3A shows an upper perspective view of the device, FIG. 3B shows a bottom perspective view of the device, and FIG. 3C shows an exploded perspective view of the device;

FIG. 4 is a block diagram schematically illustrating a monitoring device according to other possible embodiments;

FIG. 5 schematically illustrates a sleep monitoring system, utilizing a monitoring device, according a possible embodiment;

FIG. 6 is a flowchart schematically illustrating a sleep monitoring session according to some possible embodiments; and

FIG. 7 schematically illustrates an actuable mattress according to some possible embodiments.

### DETAILED DESCRIPTION OF EMBODIMENTS

One or more specific embodiments of the present disclosure will be described below with reference to the drawings, which are to be considered in all aspects as illustrative only and not restrictive in any manner. In an effort to provide a concise description of these embodiments, not all features of an actual implementation are described in the specification. Elements illustrated in the drawings are not necessarily to scale, emphasis instead being placed upon clearly illustrating the principles of the invention. This invention may be provided in other specific forms and embodiments without departing from the essential characteristics described herein.

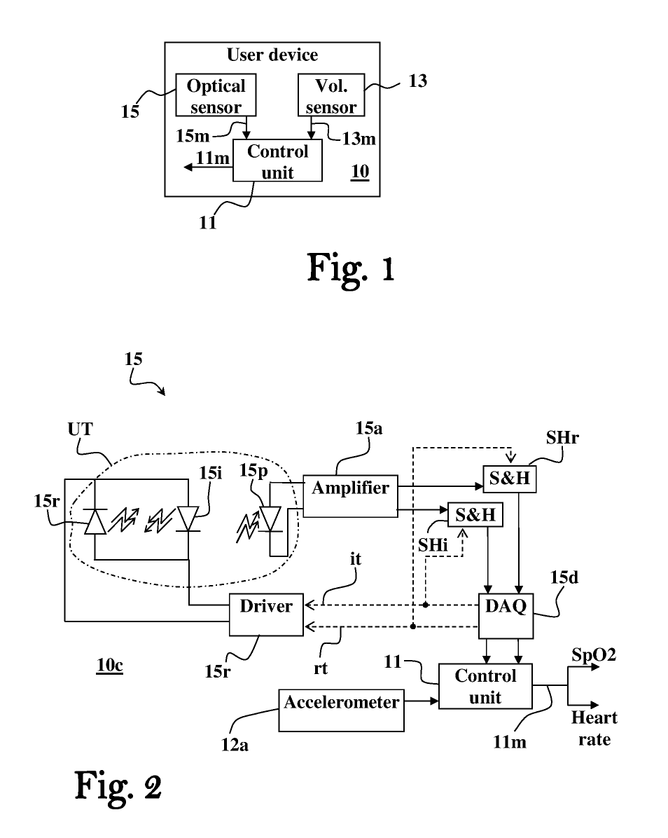

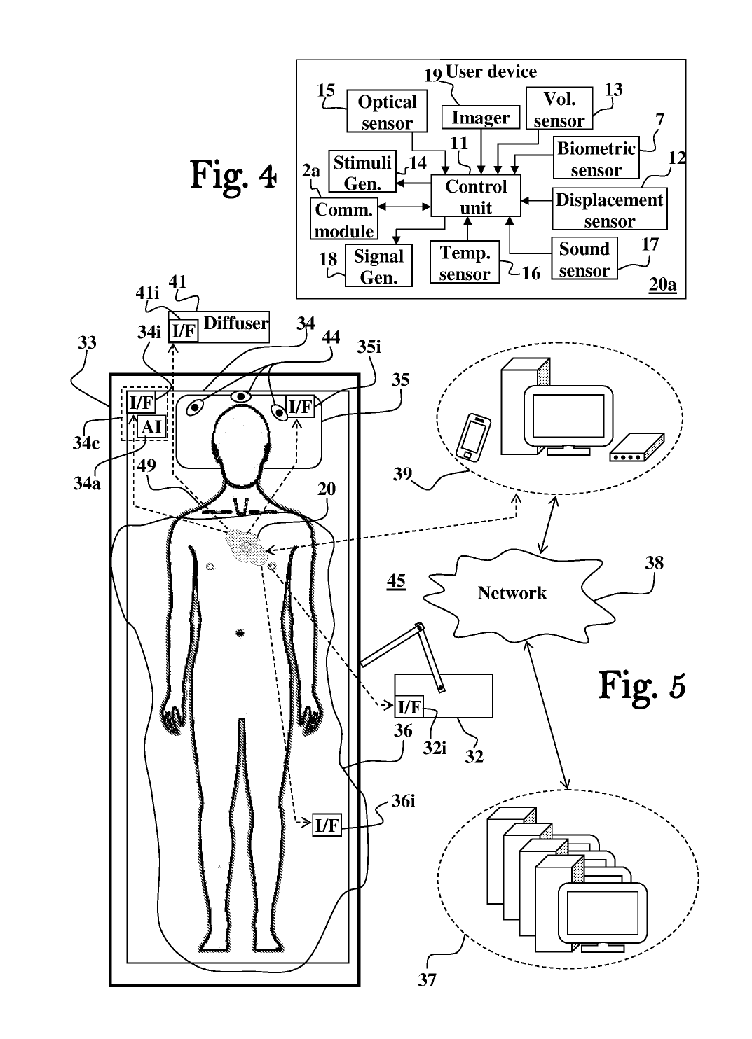

FIG. 1 is a block diagram showing a monitoring device 10 according to some possible embodiments. The monitoring device 10 is configured and arranged to be attached over a chest area of a user, measure various parameters therefrom, and generate output data 11m indicative of a sleep state or pattern of the user. The monitoring device comprises an optical sensor assembly 15, a chest volume sensor assembly 13, and a control unit 11. Optionally, and in some embodiments preferably, the monitoring device also comprises a sound transducer (microphone 17 in FIG. 4), configured and arrange to sense sounds (snores) generated by the body of the user and generate auditory data and/or signals indicative thereof.

The optical sensor assembly 15 is configured and operable to apply optical signals onto body tissues of the user, measure reflectance of the applied optical signals from the illuminated body tissues, and generate optical measurement data and/or signals 15m indicative thereof. The chest volume sensor assembly 13 is configured and operable to measure expansions and retractions of the user's chest, and generate volumetric measurement data and/or signals 13m indicative of volumetric changes in the user's chest. The control unit 11 is configured to process the optical and volumetric measurement data, and optional auditory, determine sleep state, sleeping pattern, and/or sleeping disorders, of the user, and generate output data 11m indicative thereof.

Optionally, and in some embodiments preferably, the control unit 11 is configured and operable to determine based on the optical measurement data 15m peripheral capillary Oxygen saturation (SpO2) in the blood of the user. The control unit 11 is also configured in some embodiments to determine heart pulse rate of the user based on the optical measurement data 15m. Respiratory rate on the user can be also determined by the control unit 11 based at least on one of the optical measurement data 15m from the optical sensor assembly 15 and the volumetric measurement data 13m from the volumetric sensor assembly 13.

Optionally, and in some embodiments preferably, the monitoring devices disclosed herein are provided in a form of an adhesive patch, that can be attached on a chest area of the user. In some embodiments, the monitoring device is attached over the upper part of the Sternal bone (an area called the Sternal Manubrium or Suprasternal notch). Alternatively, the monitoring device is attached below the Pectoralis muscle on the left anterior side of the chest. It is generally understood that positioning the monitoring device in proximity/adjacent to the user's heart advantageously facilitates improved blood Oxygen saturation measurements.

In order to accurately determine Oxygen saturation, the control unit is configured in some embodiments to process the volumetric measurement data 13m to identify interferences introduced into the optical measurement data 15m due to expansions or retractions of the user's chest. In this way, portions of the optical measurement data 15m identified as including interferences induced by chest expansion/retraction can be filtered out or ignored by the control unit 11, or alternatively rectified/filtered to minimize influence of the interferences on the Oxygen saturation measurement.

Pulse oximeters measurement device typically utilize a type of finger clip probe having one or more light emitting diodes (LEDs) configured to illuminate one side of a user's finger, and a photo detector configured to measure at the other side of the finger light transmitted through the finger tissue. The light emitted from LEDs at one side of the finger travels through the tissue, venous blood and arterial blood, and is collected at the other side by the photo detector. Typically, a significant portion of the light emitted by the LEDs is absorbed or scattered before it reaches the photo detector at the other side of the finger. The flow of blood is heartbeat induced, or pulsatile in nature so the transmitted light changes with time. Red and infrared lights are used for pulse oximetry to estimate the true hemoglobin oxygen saturation of arterial blood. Oxyhemoglobin (HbO2) absorbs visible and infrared (IR) light differently than deoxyhemoglobin (Hb), and appears bright red as opposed to the darker brown of Hb. Absorption in the arterial blood is represented by an AC signal which is superimposed on a DC signal representing absorptions in other substances like pigmentation in tissue, venous, capillary, bone, and so forth. Cardiac-synchronized AC signal is approximately 1% of the DC level. This is referred to as the perfusion index %. The SpO2 is typically calculated as the ratio-of-ratios by the following equation—

R=(ACrms of Red/DC of Red)/(ACrms of IR/DC of IR)

This model is often used in the literature in the context of medical devices. However, accurate % SpO2 is computed based on the empirical calibration of the ratio of ratios for the specific device, as follows—% SpO2=110−25×R.

Key considerations on how to obtain a good quality PPG (photoplethysmogram) signal will be described below with reference to FIG. 2, showing a monitoring device 10c according to some possible embodiments. It is noted that the PPG signal can also be used to extract the heart rate information of the user. Advanced applications with the PPG signal include vascular age, arterial stiffness index, and so forth. Irrespective of the target application, understanding the characteristics of light traveling through human body tissue is very important before one designs LED lighting and photo detector module fitting into a given space and location.

With reference to FIG. 2, two light emitting diodes, 15r and 15i, for selectively emitting light in the visible red and in the invisible infrared wavelength ranges, can be used with an optical sensor (photo diode) 15p in the optical sensor assembly 15 of the monitoring device 10c, for generating PPG signals indicative of the Oxygen saturation (SpO2). Optionally, a third LED (not shown) configured to emit light in the green wavelength range is used to reduce the artifacts exposure of the system by using a correlation of the signal from different wave length, thus considerably increasing the SNR (signal to noise ratio).

In this specific and non-limiting example, the driver circuit 15r is configured to selectively activate the red and infrared diodes, 15r and 15i, responsive to the timing activation signals, rt and it, generated by the data acquisition (DAQ) unit 15d. The LEDs 15r and 15i are connected in parallel and opposite polarities to the driver circuitry 15r, such that they may be selectively activated by reversing the polarity of the driving electrical voltage/current generated by the driver circuit 15r, responsive to the timing activation signals rt and it. The LEDs 15r and 15i are each configured to emit light onto the a chest tissue area UT of the user, and the light sensor 15p is configured to receive and measure the light reflected from the chest tissue area UT. The signals measured by the light sensor 15p are amplified by the amplifier circuit 15a (e.g., a transimpedance amplifier), and the amplified signals are outputted to the sample-and-hold units, SHi and SHr.

The operation of the sample-and-hold units SHi and SHr is triggered by the rt and it timing activation signals, thereby causing the SHi unit to sample and hold the signals amplified in response to the infrared emitter, LED 15i, and the SHr unit to sample and hold the signals amplified in response to the red emitter, LED 15r. The data acquisition unit 15d receives the infrared and red light signal samples from the SHi and SHr units, respectively, and convey them to the control unit 11. The control unit 11 processes the received signal samples and determines based thereon at least one of a user's blood Oxygen saturation measure and a user's hear rate measure.

A problem encountered in attaching a PPG sensor to the chest of the user is that the intensity of the measured refracted signals is relatively low. Thus, such refractive based measurements are more prone to artifacts (e.g., small movement will yield a large effect). In some embodiments an additional LED is used to emit the tissue area UT with light in the visible green wavelength range that will give more signal and will enable the smart control unit 11 to cope with any artifacts introduced in the optical measurements.

Alternatively or additionally, the device comprises an accelerometer 12a configured and operable to detect and measure movements of the user's body, and generate movement data indicative thereof. In this way, any artifacts induced due to movements of the user's body can be identified by the control unit 11 based on the movement data from the accelerometer 12a, which can then apply several corrections to negate the artifacts effects on the measured optical signals.

Optionally, the control unit 11 is configured and operable to calculate in real time the amount of energy the LEDs transmit, and use the calculated illumination power in conjuction with the movement data from the accelarometer to filter or negate the artifacts induced into the optical measurements, and thereby yield more stable readings eliminating the artifact.

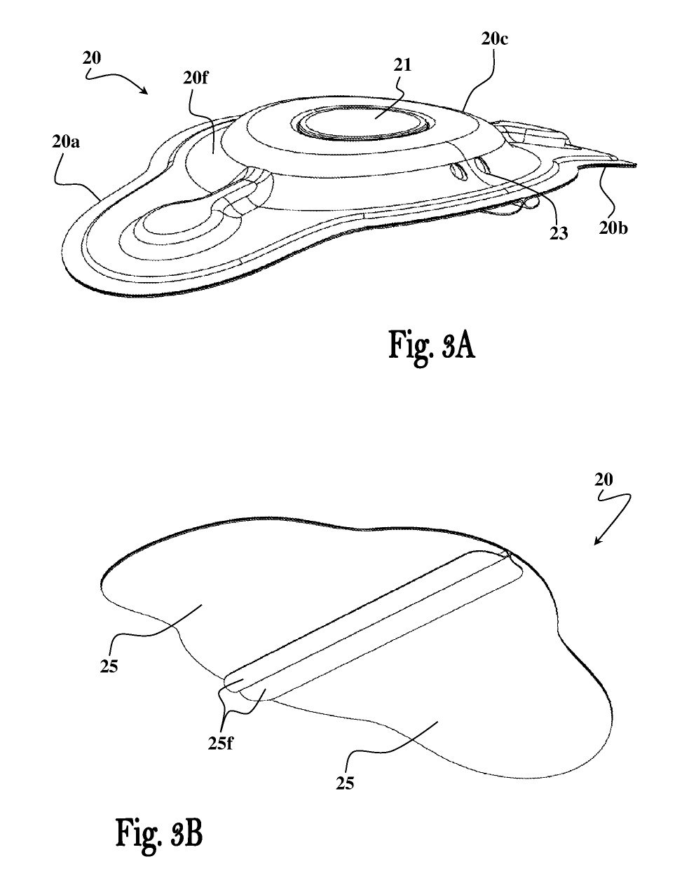

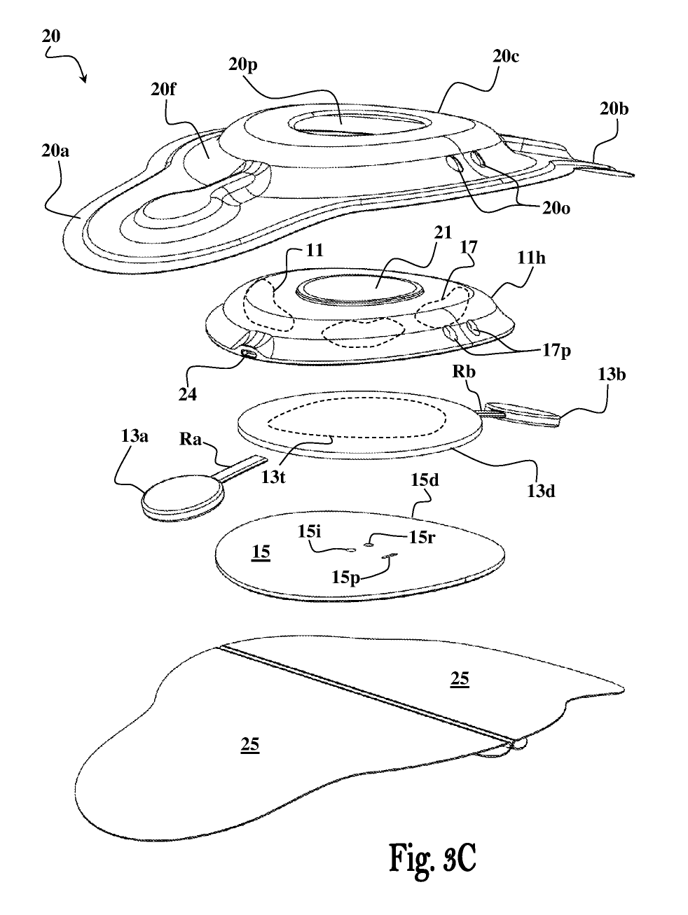

FIGS. 3A to 3C schematically illustrate a monitoring device 20 implemented according to some embodiments in a form of an adhesive patch. The monitoring device comprises two lateral elastic/deformable wings/flaps 20a and 20b extending from, and slightly tilted with respect to, a central circular base section 20c, forming a generally arc profile shape of the device 20. The wings/flaps 20a and 20b are designed to elastically move in up and down directions relative to the base section 20c. The bottom side of the monitoring device 20 comprises one or more removable cover sheets (two sheets 25 are shown in FIG. 3), which should be removed to expose an adhesive layer of the device and to thereby attach the device to the skin at the chest area of the user. The upper side of the monitoring device 20 is covered by a protective layer 20f having a central opening 20p configured to accommodate an activation button 21 of the device 20.

As seen in FIG. 3C, the circuitries of the monitoring device 20 are enclosed in a frustoconical-shaped capsule 11h accommodated in the base section 20c of the device. The activation button 21 is mounted at the top (minor base) of the capsule 11h, and configured and operable to electrically connect the circuitries of the device to a power source (not shown, e.g., disposable or rechargeable battery) of the device 20. The capsule 11h may have a data communication connector (e.g., USB, UART, or suchlike) 24 configured and operable to enable connecting the control unit 11 of the monitoring device to an external device (e.g., PC, laptop, tablet, smartphone, and suchlike). In this specific and non-limiting example one or more respective and aligned openings 20o and 17p are provided in the protective layer 20f and the capsule 11h (a pair of such opening is shown in FIG. 3), and configured to enable audio signals from the external environment of the device to propagate towards a microphone unit 17 installed in the capsule 11h.

The capsule 11h is disposed on top of the volume sensor assembly 13 that comprises an elastically deformable flat disk 13d having a tension sensor 13t mounted thereon, two elastic/bendable arms, Ra and Rb, laterally extending from the disk 13d and aligned with the wings/flaps of the device, 20a and 20b, respectively. The elastic/bendable arms Ra and Rb are slightly tilted with respect to the flat disk member 13d, and each comprises a respective contact disk member, 13a and 13b, fixedly attached to its free end. The volume sensor assembly 13 is configured to cause the contact disk members 13a and 13b to attach to the user's skin by attaching the monitoring device to the chest area of the user, and to cause deformations of the flat disk member 13d by communicating along the arms Ra and Rb the tension applied via the disk members 13a and 13b in response to chest expansions (or retractions). The tension sensor 13t is configured to sense the deformations of the flat disk member 13d and generate tension data and/or signals indicative thereof. The control unit 11 is configured to process the tension data from the tension sensor 13t and determined based thereon chest expansion and retractions events.

The flat disk member 13d of the chest volume sensor assembly 13 is mounted on top of the optical sensor assembly 15, also accommodated in the base section 20c of the device 20. The elements of the optical sensor assembly 15 are mounted on a flat disk member (e.g., printed circuit board) 15d, comprising the infrared and red LEDs, 15i and 15r, and the optical sensor 15p. The circuitries of the optical sensor assembly 15 mounted on the flat disk member 15d can comprise some or all of the elements shown in FIG. 2, and they are configured and operable to establish electrical connection with the control unit 11 mounted in the capsule 11h.

Accordingly, the monitoring device 20 can be easily deployed by the user for monitoring sleep states/patterns, simply by removing the cover sheets 25, and pressing the bottom side of the device against the skin at the chest of the user, for adhering the adhesive layer provided thereon to the skin. Once the device is properly attached at the chest area of the user, the optical sensor assembly 15 is operatively situated for measuring the PPG signals from the chest area of the user for determining the user's blood Oxygen saturation %, and the elastic/bendable arms Ra and Rb are properly deployed over the user's chest region for delivering to the disk member 13d tension/relaxations exerted due to chest expansions/retractions.

FIG. 4 is a block diagram 20a showing another possible embodiment of a monitoring device 20a. The monitoring device 20a comprises the optical sensor assembly 15 and the volume sensor assembly 13, described hereinabove with reference to FIGS. 1 to 3, and one or more additional units described hereinafter. A temperature sensor assembly 16 may be used in the monitoring device 20a to measure the body temperature of the user and generate temperature data and/or signals indicative thereof. The control unit 11 is configured in some embodiments to use the temperature data generated by the temperature sensor assembly 16 in determining of the sleep states/patterns of the user e.g., by correlating the temperature measurement data with other measurement data obtained.

Optionally, and in some embodiments preferably, the monitoring device 20a comprises a stimulation generator unit 14 configured and operable to apply sensory/mechanical (e.g., using one or more vibration motors), olfactive (e.g., using a diffuser), auditory (e.g., by one or more speakers) and/or visual (e.g., by LEDs) stimulations. A displacement sensor assembly 12 may be also used in the monitoring device 20a to measure movements of the user's body and generate movement data and/or signals indicative thereof. In this way, the control unit 11 can be configured to carry out close-loop feedback treatment sessions, by using the stimulation generator unit 14 to apply one or more stimulations to the user whenever sleep disorders are identified, and monitoring the movement data from the displacement sensor 12 to determine whether the user changed sleeping position and/or pattern in response to the applied stimulations. The control unit 11 may be configured to change parameters (e.g., intensity, frequency), and or combinations, of the applied stimulations until a change in the user's sleeping position and/or healthy sleeping patterns are determined based on the measured data.

In some embodiments the monitoring device 20a comprises a sound transducer sensor (microphone) 17 configured and operable to measure auditory signals (snore sounds) generated by the body of the user and generate auditory data and/or signals indicative thereof. The control unit 11 can be accordingly configured to process the auditory data generated by the sound sensor 17 and used in determining of the sleep state/patterns and disorders of the examined user.

The monitoring device 20a may comprise one or more imagers 19 configured and operable to generate imagery data of the user body. The control unit 11 can be configured to process the imagery data generated by the imagers 19 and determined based thereon the exact location of the monitoring device over the user's chest. If it determined by that the monitoring device 20a is not properly positioned, the control unit 11 can generate guiding instructions via the signal generator 18 (using speakers) for instructing the user to move and/or orient/rotate the device in order for it to be properly placed on the user's chest. The guiding instructions may be provided to the user using a set of LEDs and/or a by a display device provided in the monitoring device 20a (not shown, e.g., using a liquid crystal display—LCD).

Optionally, the monitoring device 20a comprises a biometric sensor unit 7 configured and operable to generate one or more biometric identifiers of the user (e.g., using user's fingerprints, facial images, and suchlike). The control unit can be accordingly configured to process the biometric identifiers generated by the biometric sensor unit 7 to authenticate that the monitoring device is attached to a legal user/owner of the monitoring device 20a.

A communication module 2a is also provided in some embodiments in the monitoring device 20a for communicating data and/or instructions with external devices (e.g., smartphone, laptop, tablet, or any other computerized device), the communication link 2a may be configured and operable to communicated data over any suitable wired (e.g., USB, UART, and suchlike) or wireless (e.g., WiFi, BlueTooth, ZigBee, NFC, IR or sonic/ultrasonic signaling) communication link and using any communication protocol suitable for this purpose.

FIG. 5 schematically illustrates a sleep monitoring system 45 utilizing the monitoring device 20 placed on the chest area of the user 49. The user 49 is seen in this example in a supine position on a mattress 34 of bed 33, with user's head resting on a pillow 35, and covered by a blanket 36. The mattress 34, pillow 35 and/or blanket 36 may comprise sensors for measuring various parameters (e.g., body temperature, position, electrical conductance/impedance, and suchlike) of the user and/or actuators for applying one or more sensory stimulations. For this purpose the mattress 34, pillow and/or blanket 36, and/or pillow/mattress cover, may each comprise a communication interface, 34i 35i and 36i, respectively, each configured and operable to communicate data/instructions with the monitoring device 20 over any suitable wired and/or wireless communication links.

As will be apparent from the following description, the monitoring device 20 of FIG. 5 can comprise any of the units/devices provided in the monitoring device 20a shown in FIG. 4.

The pillow 35 (or mattress 34) may comprise three or more speakers 44 for playing tunes, and/or binaural audio patterns and/or beats, to the user for promoting healthy sleep patterns and provoking change in sleep position, if so needed. The monitoring device 20 can be configured to generate audio streams to be played to the user through the speakers 44 during a treatment session. The audio streams generated by the monitoring device 20 may be binaural signals and/or comprise binaural beats. The control unit of the monitoring device can configured to adjust the frequency the binaural signals to a respiratory or heart rate of the examined user 49. The monitoring device may be also configured to selectively add or remove one or more speakers in the generated audio stream according to the body position of the body of the user to maintain continuous stereo play of the generated audio data regardless of the whether the user is in a supine, side or abdomen position.

In some embodiments one or more robotic arm units 32 are used in the system to facilitate change in the body position of the user 49. The robotic arm units 32 can be positioned beside the bed 33, or attached to the bed 33, and configured and operable to communicate data/instructions with the monitoring device 20 via a dedicated communication interface 321 thereof. In this way whenever sleep disorders are identified, the monitoring device 20 can instruct one or more robotic arms 32 to contact, and/or move, the user's body for causing a change in the body position for restoring healthy sleep patterns (e.g., by causing opening of air paths). Each robotic arm 32 may comprise contact and/or pressure sensors (not shown) configured for controlling the amount of contact/pressure applied by the robotic arm 32 to the body of the user 49.

The system 45 may comprise a diffuser 41 configured and operable to generate olfactory stimuli and/or evaporate certain drugs/medicaments for provoking the user to cause change in the user's body position, and/or for promoting healthy sleep patterns. The diffuser is configured to receive signals and/or instructions from the monitoring device via a dedicated communication interface 411 embedded therein. It is note that the diffuser 41 may be provided in any of the elements of the system 45, and/or as an independent unit. For example, in some possible embodiments a diffuser 41 is provided as part of the robotic arm unit 32.

The monitoring device 20 may be configured to communicate data/instructions with computerized device 39 (e.g., smartphone, personal computer—PC, router) in the vicinity/same room of the user 49. In this way the monitoring device 20 can receive user input data, and transfer data accumulated over time to a remote database and/or monitoring center 37, over a data network (e.g., the Internet). This can be advantageously employed for use of disposable monitoring devices 20, by loading the accumulated data about the sleeping patterns and/or disorders, and the stimulation found to be effective in promoting healthy sleeping patterns, into a new monitoring device 20, whenever a used device 20 is being disposed of.

In some embodiments the communication interface 34i of the mattress 34 is part of a removable clip device 34c configured to collect the measured data from one or more, or all, of the sensor units/assemblies of the system. The clip device 34c can comprise a separate control unit (not shown) configured and operable to process the measurement data and generate clinical diagnostics therefrom. The clip device 34c can be configured to transmit via the communication interface 34i instructions to the speakers 44 to play audio and/or beat signal, with or without binaural patterns, and/or to the one or more robotic arm units 32 to contact/move the user's body, and/or to the diffuser to diffuse substances into the air, and/or to any other actuator unit is the system for applying stimulations to the body of the user 49.

The clip device 34c comprises in some embodiments an artificial intelligence (AI) module 34a having learning capabilities and engineered to identify over time the stimulations, and/or combination of stimulations, and their frequencies and/or intensities, that successfully managed to cause the user 49 to change sleep position and/or gain healthy sleep states. The AI module 34a may be configured and operable to adjust the audio signals and/or any other stimulation applied to the user 49 convey subliminal message and/or prescription audio therapy. The clip device 34c may be configured and operable to communicate the diagnostic data, and/or measure data, and/or the treatment data accumulated therein over time to any of the computerized devices 39 and/or the remote database/monitoring center 37.

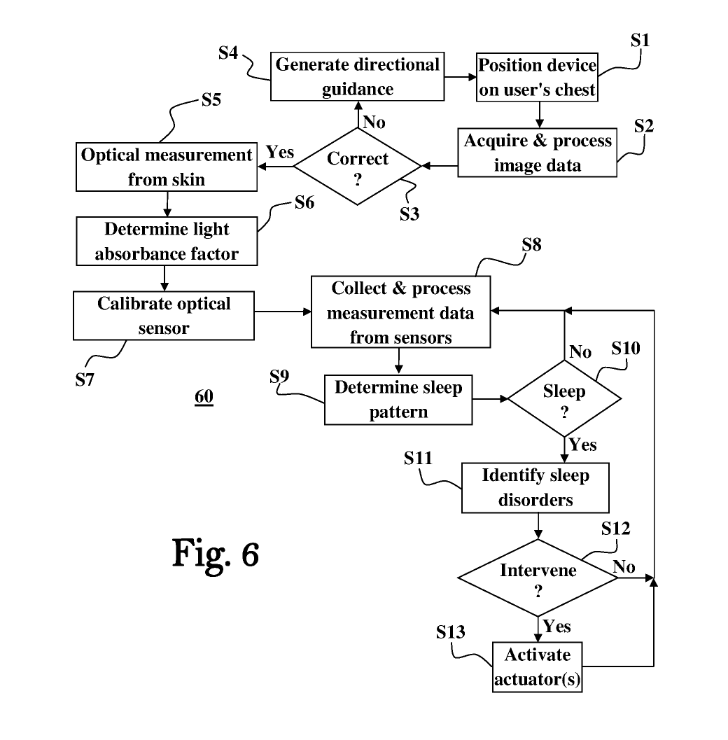

FIG. 6 shows a flowchart 60 schematically illustrating a sleep monitoring/treatment session according to some possible embodiments. The session starts in step S1 by positioning the monitoring device over the user's chest. Next, in step S2, image data generated by the monitoring device is processed for determining in step S3 if the monitoring device is properly positioned on the user's chest. If the monitoring device is disoriented and/or dislocated, in step S4 directional guidance/instructions are generated by the device to instruct the user how to correct the orientation and/or location of the device over the user's chest.

Once the monitoring device is properly located on the user's chest, in step S5 optical measurements on the user's skin are performed, and in step S6 the performed measurements are used to determine a light absorbance factor of user's skin. In step S7, the light absorbance factor determined in step S6 is used to calibrate the optical sensor of the monitoring device, to thereby improve the accuracy of the measure optical data by adjusting the intensity of the emitted light to the color/pigment of the user's skin. After calibrating the optical sensor, in step S8 the monitoring device starts collecting measurement data from the different sensor units, and in step S9 the collected measurement data is processed to determine sleep patterns of the examined user.

In step S10 it is checked if the sleep patterns determined in step S9 are indicative of the user being in a sleeping state. If it is determined that the user is not in a sleeping state, the control is passed back to steps S8 and S9 for continuously or periodically collecting new measurement data and determining new sleep patterns of the user. Whenever it is determined that the user is in a sleeping state, in step S11 the sleep state/patterns are processed to determine if the user is experiencing any sleep disorders, and in step S12 the device determines if the sleep disorders require intervention. If it is determined in step S12 that there is no need for external intervention, the control is passed back to step S8 to S11 for continuously or periodically collecting new measurement data and determining if newly determined sleep patterns requires intervention.

If it is determined in step S12 that external intervention is required for changing the user's body position and/or sleeping pattern, in step S13 one or more actuators are activated for generating stimuli to cause the user to change body position. The control is then passed back to step S8. In this way, measurement data can be continuously collected from the user's body, while the system monitors the sleeping state of the user and generates stimulation(s) whenever needed to guarantee that unhealthy sleep patterns be rectified to gain healthy sleep patterns, substantially without awakening the user.

It should also be understood that throughout this disclosure, where a process or method is shown or described, the steps of the method may be performed in any order or simultaneously, unless it is clear from the context that one step depends on another being performed first.

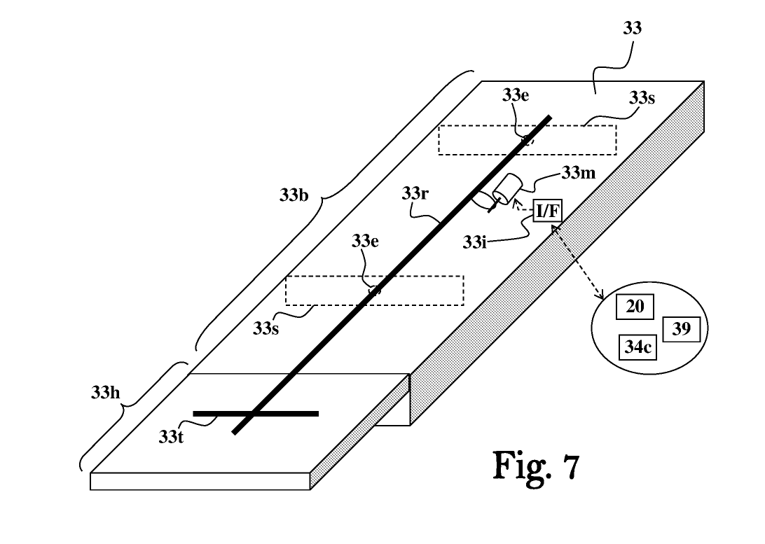

FIG. 7 schematically illustrates an actuable mattress 33 used according to some possible embodiments in the system 45 (show in FIG. 5). The mattress 33 generally comprises a main body base section 33b, configured to comfortably accommodate user's torso and upper and lower limbs, and a tiltable heat support section 33h, having smaller thickness/dimension than that of the base section 33b and extending from frontally from the based section 33b. The head support section 33h may be an integral part of the based section 33b, or it may be configured as a separate part attached, or mounted adjacent, to a frontal side of the mattress 33.

The mattress 33 comprises an elongated rotating rod 33r passing along a length of the mattress 33 and extending at least partially into the heat support section 33h. One or more transversal support elements 33s may be provided in the mattress 33 configured and arranged to support the rod 33r thereinside, prevent translational movement of the rod 33r thereinside while facilitating its rotational movement e.g., by bearings 33e provided in the support elements 33s. Motor unit 33m mechanically coupled to the rod 33r (e.g., via transmission belt and/or gear wheels) is configured and arranged to rotate the rod 33r responsive to instructions received via the communication interface 331 of the mattress 33. The head support section 33h comprises a transversal actuating bar 33t, attached to the rod 33r and traversing a certain width of the head support section 33h. The motor unit 33m is configured in some embodiments to apply sequences of reciprocating rotations i.e., rotations and counter-rotations pulses, at determined frequencies and/or velocities to cause the head support section 33h to roll about the axis of the rod 33r.

The communication interface 331 may be configured to communication data and/or instructions over wired, or wireless, communication links, with the monitoring device 20, any computerized device 39, and/or the mattress clip device 34c.

Functions of the system described hereinabove may be controlled through instructions executed by a computer-based control system which may be housed in the monitoring device, the clip device, and or other elements of the system having data communication capabilities. A control system suitable for use with embodiments described hereinabove may include, for example, one or more processors connected to a communication bus, one or more volatile memories (e.g., random access memory —RAM) or non-volatile memories (e.g., Flash memory). A secondary memory (e.g., a hard disk drive, a removable storage drive, and/or removable memory chip such as an EPROM, PROM or Flash memory) may be used for storing data, computer programs or other instructions, to be loaded into the computer system.

As will be appreciated by one skilled in the art, aspects of the present invention may be embodied as a system, method or computer program product. Accordingly, aspects of the present invention may take the form of an entirely hardware embodiment, an entirely software embodiment (including firmware, resident software, micro-code, etc.) or an embodiment combining software and hardware aspects that may all generally be referred to herein as a “circuit,” “module” or “system.” Furthermore, aspects of the present invention may take the form of a computer program product embodied in one or more computer readable medium(s) having computer readable program code embodied thereon.

To illustrate the interchangeability of hardware and software, items such as the various illustrative blocks, modules, elements, components, methods, operations, steps, and algorithms have been described generally in terms of their functionality. Whether such functionality is implemented as hardware or software depends upon the particular application and design constraints imposed on the overall system. Skilled artisans may implement the described functionality in varying ways for each particular application.

As described hereinabove and shown in the associated figured, the present invention provides sleep monitoring devices, systems and actuators used thereby for monitoring and diagnosing sleep patterns of a user, and related methods for treating unhealthy sleep patterns. While particular embodiments of the invention have been described, it will be understood, however, that the invention is not limited thereto, since modifications may be made by those skilled in the art, particularly in light of the foregoing teachings. As will be appreciated by the skilled person, the invention can be carried out in a great variety of ways, employing more than one technique from those described above, all without exceeding the scope of the claims.

1. A device for monitoring one or more parameters of a subject, the device comprising:
a support element configured and arranged to detachably attach over a skin region of a chest area of a subject;
an optical sensor assembly mounted to said support element and configured and arranged to conduct optical measurements in said chest area and generate optical measurement data indicative of said one or more parameters of said subject;
a volumetric sensor assembly mounted to said support element and configured and operable to measure chest expansions and retractions of said subject and generate volumetric measurement data indicative of the expansions and retractions of the chest of said subject; and
a control unit mounted to said support element and configured and operable to process said optical and volumetric measurement data for removing interferences introduced into said optical measurements due to said chest expansions and retractions.

2. The device of claim 1 comprising a displacement sensor assembly configured and operable to measure displacements of said device and generate displacement measurement data indicative thereof.

3. The device of claim 1 wherein the control unit is configured and operable to determine breath rhythm of the monitored subject based on at least one of the displacement measurement data and the volumetric data.

4. The device of claim 1 wherein the one or more parameters comprises oxygen saturation.

5. The device of claim 1 wherein the control unit is configured and operable to determine heart rate of the subject based on the optical measurement data.

6. The device of claim 1 configured and operable to measure respiration or breathing rate.

7. The device of any claim 1 comprising a sound measurement assembly mounted to the support element and configured and operable to measure respiratory sounds of the subject and generate data indicative thereof.

8. The device of claim 1 wherein the control unit is configured and operable to determine sleep, sleep architecture, sleep pathologies, environment influence.

9. The device of claim 1 wherein the displacement sensor assembly comprises an accelerometer.

10. The device of claim 8 comprising a stimulation arrangement mounted to the support sheet and configured to apply at least one stimuli to the subject based on determined sleep pattern and/or state.

11. The device of claim 10 wherein the stimulation arrangement comprises at least one of the following: electrodes mounted to the support element and configured and arranged to apply electrical stimulation to the skin region of the subject electromechanical assembly mounted to the support element and configured and arranged to apply vibrations to the skin region of the subject signals generator mounted to the support element and configured and arranged to produce auditory signals; an evaporator and/or diffuser mounted to the support element and configured and arranged to discharge one or more odorants and/or drugs and/or visual stimulation.

12. The device of claim 10 wherein the stimulation arrangement comprises a mechanical actuator assembly mounted to the support element and configured and arranged to apply tactile pressure or vibratory stimulations.

13. The device of claim 10 comprising a position sensor assembly mounted to the support sheet and configured and operable to generate position measurement data indicative of a body position of the subject.

14. The device of claim 13 wherein the control unit is configured and operable to identify sleep disorders or sleep architecture and responsively use the stimulation arrangement to apply one or more stimuli to the subject.

15. The device of claim 14 wherein the control unit is configured and operable to apply the one or more stimuli until the position measurement data is indicative of a change in a body position of the subject.

16. The device of claim 14 wherein the audio signals generator comprises at least two speakers, and wherein the control unit is configured and operable to cause said audio signals generator to produce directional auditory stimuli to cause the subject to change body position.

17. The device of claim 15 wherein the control unit is configured and operable to adjust at least one of frequency and magnitude of at least one of the applied stimuli according to the measured data and guarantee that a continuous sleep state of the subject is maintained, or to cause a light sleep state thereof without awakening the subject.

18. The device of claim 17 wherein the control unit is configured and operable to synchronize between at least one of the measured parameters and at least one of the applied stimuli.

19. The device of claim 14 wherein the control unit is configured and operable to apply the one or more stimuli until identifying that the sleep disorder has lapsed and a healthy sleep pattern been established.

20. The device of claim 10 wherein the control unit is configured and operable to use the stimulation arrangement to apply sleep aiding stimulations whenever identifying that the subject awakened until sleeping patterns are restored.

21. The device of claim 13 coupled to an external actuator configured and operable to mechanically move one or more regions and/or organs of the subject to thereby affect a change in the body position of the subject, and wherein the control unit is configured to actuate said external actuator whenever sleep disorders are identified.

22. The device of claim 21 wherein the external actuator comprises at least one robotic arm.

23. The device of claim 1 comprising a communication unit configured and operable to exchange data and/or instructions with said device via RF and/or sonic/ultrasonic and/or optical data communication.

24. The device of claim 23 wherein the control unit is configured and operable to use the communication unit to communicate data over a data network with at least one remote computer device or server.

25. The device of claim 23 wherein the control unit is configured and operable to use the communication unit to communicate data with at least one user device.

26. The device of claim 23 wherein the control unit is configured and operable to carry out calibration of the device based on data inputs received from the subject via the communication unit.

27. The device of claim 26 wherein the data inputs comprise at least one of the following: subject's weight, subject's height, subject's BMI, subject's skin color, subject's age, subject's geographic location, subject's gender, subject's general ethnic classification, subject's body fat mass.

28. The device of claim 26 wherein the calibration comprises adjusting operational parameters of the optical sensor assembly based on the received data.

29. The device of claim 1 comprising at least one imaging unit configured and operable to generate imagery data of the subject.

30. The device of claim 25 wherein the control unit is configured and operable to receive and process imagery data received from at least one of the imaging unit and/or the at least one user device and determine the location of the device based thereon.

31. The device of claim 30 wherein the control unit is configured and operable to determine location of the device relative to upper sternum of the subject, or to anterior chest on the left side below the pectoralis muscle.

32. The device of claim 31 wherein the control unit is configured and operable to generate directions instructing precisely relocating the device over a desired are of the chest of the subject.

33. The device of claim 1 comprising a temperature measurement unit configured and operable to measure body temperature of the subject and generate temperature data indicative thereof.

34. The device of claim 1 wherein the control unit is configured and operable to determined Spo2 of the subject based on the measured data.

35. The device of claim 1 configured and operable to measure electrocardiogram and/or electromyogram and/or electroencephalogram of the subject.

36. The device of claim 1 comprising one or more biosensors configured and operable for measuring biochemical components in the skin, or through the skin, or in the blood, of the subject.

37. The device of claim 36 wherein the biochemical components comprise at least one of the following: hormones; and/or neurotransmitters; and/or toxins; and/or drugs; and/or pathogens; and/or any precursor of molecules of these biochemical components and/or any degradable molecule that is the result of these biochemical components.

38. The device of claim 1 comprising at least one biometric sensor arrangement configured and operable to measure at least on biometric property of the subject and generate data indicative thereof.

39. The device of claim 38 wherein the control unit is configured and operable to identify the subject based on the data generated by said at least one biometric sensor arrangement.

40. The device of any claim 1 wherein the control unit is configured and operable to determine at least one of apnea hypopnea index (AHI) and respiratory disturbance index (RDI) of the subject based on the measured data.

41. A method of monitoring sleep state of a subject, comprising: measuring optical data indicative of passage of light through tissue at a chest area of said subject, measuring tension data indicative expansions and/or retractions of the subject's chest, and processing said optical and tension data to determine a sleep pattern and/or state of the subject.

42. The method of claim 41 comprising processing the tension to identify interferences induced in the measured optical data, and manipulating said optical data to minimize effects of said interferences thereon.

43. The method of claim 41 comprising processing the measured optical data to determine at least one of blood Oxygen saturation, breathing rate, heart rate.

44. The method of claim 41 comprising processing the measured tension data to determine at least one of breathing rate of the subject, and chest expansion events, chest retraction events.

45. The method of claim 41 comprising measuring at least one sound data indicative of sounds generated by the subjects, imagery data of the body of the subject, temperature data indicative of body temperature of the user.

46. The method of claim 41 comprising processing the measured data to determine one or more sleep patterns of the subject, and applying one or more stimulations whenever identifying unhealthy sleep patterns in the determine one or more sleep patterns.

47. The method of claim 46 wherein the one or more stimulations comprises at least one of binaural audio signal and beats.

48. A system for monitoring sleep of a subject, the system comprising; a monitoring device configured and arranged to attach to a chest area of the subject, said monitoring device being configured and operable to measure optical data and tension data from the chest area of the subject and determine at least one sleep pattern of said subject based thereon, and at least one stimulation applicator configured and operable to apply one or more stimulations to the subject whenever unhealthy sleep patterns are identified.

49. The system of claim 48 wherein the at least one stimulation applicator comprises at least one of three or more speakers, one or more robotic arms, one or more electrodes, one or more vibrators, one or more diffusers, and an actuable mattress.

50. The system of claim 49 wherein the actuable mattress comprise a movable head support section and an actuator configured and operable to apply rotary movement to said head support section.
